(19) United States
(12) Patent Application Publication
(10) Pub. No.: US 2017/0260563 A1
(43) Pub. Date: Sep. 14, 2017

(54) TESTING TOXICITY OF A TEST SAMPLE

(71) Applicant: ABOATOX OY, MASKU (FI)

(72) Inventor: Juha Lappalainen, Masku (FI)

(73) Assignee: ABOATOX OY, MASKU (FI)

(21) Appl. No.: 15/511,028

(22) PCT Filed: Sep. 15, 2015
(86) PCT No.: PCT/FI2015/050617

§ 371 Date: Mar. 14, 2017

(30) Foreign Application Priority Data
Sep. 16, 2014 (FI) 20145810

(51) Int. Cl. C12Q 1/04; G01N 21/76; G01N 33/52

(52) U.S. Cl. CPC C12Q 1/04; G01N 33/528; G01N 21/76; G01N 2333/195; G01N 2021/751

(57) ABSTRACT

Invention describes a method and a device for testing toxicity of a test sample. The method comprises at least the steps of: providing an aliquot or a volume of light producing test reagent liquid in a reagent vial, moistening a control swab with the light producing test reagent liquid, moistening a test swab with the test sample, moistening the test swab with the light producing test reagent liquid, reading luminescence of the control swab in a luminometer and thereby obtaining a luminescence value of a control sample, reading luminescence of the test swab in the luminometer and obtaining a luminescence value of the test sample, calculating the toxicity of the test sample by comparing the luminescence value of the control sample to the luminescence value of the test sample. According to the invention the moistenings of the control swab and the test swab with the light producing test reagent liquid are done with the same aliquot of light producing test reagent liquid in the same reagent vial.”

### TECHNICAL FIELD OF THE INVENTION

The invention relates to a method and device for testing toxicity of a test sample. The invention especially relates to a new way of obtaining the toxicity of the test sample by comparing a luminescence value of a control sample in a control swab to a luminescence value of a test sample in a test swab.

### TECHNICAL BACKGROUND

Luminescent bacteria or other light producing test reagents are widely used for the determination of toxicity from water samples. In the ISO Standard method 11348-3, a vial of the lyophilized reagent is taken from a freezer and the reagent is reconstituted with reconstitution solution. The reconstituted reagent is divided into aliquots. These aliquots are mixed with test samples and controls samples in test tubes. After a predetermined contact time the test tubes are transferred to a luminometer and measured. The control sample result is compared to the test result. Therefore, it is convenient to have several samples and sample dilutions (10-100) that are measured in a series at the same time because the reconstituted reagent must be used during the same day after reconstitution. The reagent cost for one test sample only is he same as it would be for 100 samples. The use of the system is so complicated due to several pipeting steps that it is not convenient to perform it in the field conditions outside the laboratory.

### OBJECT OF THE INVENTION

It is an object of the present invention to reduce or even eliminate the above-mentioned problems appearing in prior art.

An object of the present invention is to provide for a simple and cost-effective testing of toxicity of a sample by comparing luminescence values of a control sample and a test sample.

An object of the present invention is to provide for a new way to use swabs in testing of toxicity of a sample.

### SUMMARY OF THE INVENTION

Among others, in order to realize the objects mentioned above, methods, devices, uses and other objects according to the invention are characterized by what is presented in the characterizing parts of the enclosed independent claims.

The embodiments, examples and advantages mentioned in this text relate, where applicable, to any methods, devices or uses according to the invention, even though it is not always specifically mentioned.

A typical method for testing toxicity of a test sample according to the invention comprises at least following steps
providing an aliquot or a volume of light producing test reagent liquid in a reagent vial
moistening a control swab with the light producing test reagent liquid
moistening a test swab with the test sample
moistening the test swab with the light producing test reagent liquid
reading luminescence of the control swab in a luminometer and thereby obtaining a luminescence value of a control sample
reading luminescence of the test swab in the luminometer and obtaining a luminescence value of the test sample
calculating the toxicity of the test sample by comparing the luminescence value of the control sample to the luminescence value of the test sample;

whereby the moistenings of the control swab and the test swab with the light producing test reagent liquid are done with the same aliquot of light producing test reagent liquid in the same reagent vial.

A typical device for testing toxicity of a test sample according to the invention comprises a device body and at least one vial attached to said device body. The device body may be elongated. A device body may comprise:
a first end with a swab opening for inserting a control swab and/or a test swab into the device body and for removing the swabs from the device body
a second end with vial fastening means for releasably attaching a vial to the elongated body,
at least two swab tubes inside the device body, where at least one swab tube is adapted to receive the control swab and at least one swab tube is adapted to receive the test swab.

The at least one vial is adapted to be releasably attached to said vial fastening means.

One advantage of the invention is its simplicity. Two measurement results, control result and test result, are determined from one single dose of reagent.

One advantage of the invention is that the swabs may be kept, e.g. transported, and the luminescence may be measured, in any position. There is no vial or other vessel with a liquid phase to be measured. The liquid needed for the test stays easily in the swab heads. The measurements can be done with any luminometer where the device fits, also with a plate reader luminometer with a special insert or plate.

The device body is easy to design in such a way that the device can be put into a luminometer with both the swabs inside the device. Some examples (trademarks) of possible luminometers that may be used with the present invention are: Kikkoman PD10, Kikkoman PD20, 3M Clean-Trace, Hygiena System Sure Plus, Charm novaLUM, Merck Hy-Lite 2, Titertek FB14. Luminometers are known as such and not described further here.

In an embodiment the test sample and the aliquot of light producing test reagent liquid are mixed together, thereby producing a mixture, and the test swab is moistened in said mixture. This way the two moistenings of the test swab may be done simultaneously, in one step.

In an embodiment the swab tubes are arranged to keep the swabs in them separated from each other and arranged to allow moving the swabs inside the swab tubes.

In an embodiment the device comprises at least one control swab and at least one test swab. The device is adapted to allow the control swab and the test swab to be moved in the swab tubes between at least two positions, i.e.:
a down position, where an end of the swab is at least partly inside the vial attached to the vial fastening means
an up position, where the end of the swab is not inside the vial attached to the vial fastening means.

In an embodiment the control swab and the test swab are moved inside the elongated device body by contacting them through one or more swab moving openings formed at the side of the elongated device body.

In an embodiment the light producing test reagent is reconstituted with reconstitution liquid in the reagent vial. The reconstituting may be done in various different ways. One way is to provide a reconstitution liquid chamber, e.g. a small bag or vial with a specific aliquot or volume of reconstitution liquid in it, in or above the reagent vial and puncture the chamber e.g. with the control swab. This would cause the reconstitution liquid to mix with the light producing test reagent in the reagent vial. Reconstitution can also be done e.g. outside the device by pouring known amount of the liquid to the vial before attaching it to the device.

In an embodiment the device comprises one or more of the following vials and in an embodiment the method comprises attaching to and/or removing from the vial fastening means at least one of the following:
the reconstitution liquid chamber
the reagent vial containing test reagent liquid
an empty reagent vial
a vial containing dried light producing test reagent such as freeze dried bacteria
a measurement vial for protecting the luminometer during the reading of luminescence.

In an embodiment at least part of the control swab and/or the test swab is inserted in to a vial attached to the vial fastening means. This enables e.g. moistening of the swab head with any liquid present in the vial.

In an embodiment the light producing test reagent is a luminescent organism or a luminescent bacteria, such as natural bacteria, e.g. Aliivibrio fischeri or genetically modified bacteria or yeast producing light or other luminescent organism and that the luminescent organism is selected from: freeze dried organism, liquid dried organism, fresh organism, freeze dried bacteria, liquid dried bacteria or fresh bacteria.

In an embodiment at least one vial comprises luminescent bacteria as a light producing test reagent.

In an embodiment the light producing test reagent is selected from
bioluminescence from ATP reaction
chemiluminescence
bacterial luminescence.

In an embodiment the reading of luminescence of the control swab and the test swab are made in the luminometer directly from the swabs. Measuring directly from the swabs means that no vial with a substantial amount of liquid is present around the moist swab heads during the reading of luminescence. In other words, at least substantially all, preferably all liquid present is absorbed in the swabs during the reading of luminescence of the control swab and the test swab. This way there is no liquid that can spill and contaminate the measurement chamber of the luminometer, e.g. in the case where the user forgets to remove the device from the luminometer. With portable systems this is a risk that can lead to false results and laborious cleaning process.

In an embodiment a swab head made of 100% polyester is used for testing toxicity of a test sample. In one embodiment the handle of the swab is made of polypropylene. In one embodiment the polyester head is constructed to the handle without adhesives. If the swab is used as a test swab it is moistened with a light producing test reagent liquid and the test sample. A control swab is moistened with only the light producing test reagent liquid. Luminescence of the swabs is measured in the luminometer directly from the swab, i.e. at least substantially all, preferably all liquid present is absorbed in the swabs during the reading of luminescence. There is no need to measure the luminescence from a liquid phase.

In an embodiment the control swab and the test swab comprise swab heads in only one end of their swab handle.

In an embodiment a swab comprises two swab heads, one on each end of the swab handle. A swab with two swab heads may be used so that one swab head functions as a control swab and the other swab head as a test swab. This way only one swab is needed for the invention.

According to one embodiment of invention, one test sample result and one control result are obtained with only one vial of reagent. With a simple device it is made possible to test toxicity in field conditions outside the laboratory even without pipeting of liquids. All components needed for the test, i.e. reagent, reconstitution solution and swabs for the liquid transfer, may be packed in the simple device. The control sample is taken from the reconstituted bacteria reagent with an inert swab. The test sample is taken with another swab and brought to contact with the same reconstituted bacteria reagent, or the test sample can be added directly to the bacteria reagent and an aliquot is taken from this suspension with the swab. Both swabs are then incubated inside the device and the luminescence is measured from both swabs separately. The inhibitory effect of the test sample on the luminescent bacteria is calculated by comparing the luminescence values from the unaffected bacteria (i.e. control sample) to the luminescence values from the test sample. The aliquots or volumes of liquid and bacteria transferred with the swab are constant enough for this application. Because the swab material is inert the control result is comparable to non-stressed bacteria.

According to one embodiment of invention the control and test swabs may be inserted in the device in such a way that during the reading of luminescence the swab heads point towards opposite directions. E.g. the test control head may point up and the test swab head may point down. This way the swab tubes inside the device are not needed for separating the swabs from touching each other if the swab handles are long enough. A swab with two swab heads may also be used: One swab head as a control swab and the other swab head as a test swab. With this method it is possible to use a device which comprises no swab tube or only one swab tube inside the device body.

An embodiment of the invention comprises two readings of luminescence, where one reading comprises reading luminescence of both the control swab and the test swab in a luminometer and thereby obtaining a total luminescence value of both swabs. The other reading comprises reading luminescence of only one of the control swab and the test swab thereby obtaining luminescence value of a control sample or test sample. By subtracting luminescence value of the control or the test sample from the total luminescence value of both swabs the luminescence value of the other sample is obtained. These values can be used to determine toxicity of the test sample.

In one embodiment, first total luminescence value of both swabs is read, thereafter control swab is removed, thereafter luminescence value of the test sample is read. One possible criterion for determining toxicity is that if the luminescence value of the test sample is less than 50% of the total luminescence value of both swabs, the sample is toxic to the bacterium used.

The invention may be used with a plate luminometer and with a special insert or plate for a plate luminometer. Such an insert for a plate reader luminometer has outer diameters of a typical plate, e.g. a plate with 96 wells. Instead of the many wells in a normal plate, there is one or more larger opening or recess where one or more devices of the invention fit. The plate reader can e.g. be programmed to automatically measure luminescence at all the possible swab head locations in the insert. The openings or recesses in an insert for a plate luminometer may be adapted to receive any of the devices presented in this text, whether with one or two swabs or with none, one or two swab tubes inside the device.

In an embodiment an insert or a plate for a plate luminometer comprises a top face and a bottom face. The device with the swabs is arranged into the one or more opening or recess through the top face. The bottom face may comprise one or more light openings or windows for passage of emitted light from a swab arranged in said one or more opening or recess.

In an embodiment the plate reader is capable of reading luminescence from the swabs from both sides of a device, an insert or a plate, possible simultaneously.

In an embodiment the plate reader is capable of reading luminescence from one side only, but a device or an insert or a plate has different swabs visible towards two different sides. E.g. for this purpose the luminescence can first be read from the first side, and the device or the insert or plate can be turned upside down for reading luminescence of the swabs on the second side. The device or the insert or plate may thus be adapted to be situated into the luminometer in at least two different positions. E.g. an insert or a plate for a plate luminometer may comprise suitable positioning shapes or devices to allow it to be situated in two positions into the luminometer.

The device body with the swab tubes and vials may be made of various materials. At least some parts of the device may be transparent in order to allow reading luminescence through them. The device body or vial, e.g. the measurement vial may be a cuvette. There are international standards for the dimensions and material for cuvettes. They can be made e.g. of a plastic, having e.g. round or rectangular cross-section with e.g. one centimeter diameter.

In one embodiment of the invention, the device comprises a non-transparent partition wall between the swabs in the device. This makes it possible to read luminescence from the swabs from both sides, even simultaneously.

It is possible that the device body functions as the measurement vial. It this case the device according to the invention doesn't necessary need any vial fastening means or an at least one vial attached to said device body.

The swab heads may be made from 100% polyester. One possible swab type to be used is Texwipe's Alpha® Swab Series. The swab head may be made from 100% polyester knit materials. Complete thermal bond construction of the swab eliminates adhesive contamination. Double layer of polyester knit fabric enhances absorbency. 100% virgin polypropylene handle ensures no additional contaminants are introduced. The swabs may even be cleanroom laundered.

### BRIEF DESCRIPTION OF THE FIGURES

The invention is described in more detail below with reference to the enclosed schematic drawing, in which

FIGS. 1a, 1b and 1c show a device and its parts according to the invention,

FIGS. 2-8 show a device according to the invention in some stages of a method according to the invention,

FIGS. 9a and 9b show a second device according to the invention,

FIG. 10 shows a third device according to the invention,

FIG. 11 shows a device according to the invention in a plate of a plate reader,

FIGS. 12a and 12b show a fourth device according to the invention,

FIG. 13 shows a plate of a plate reader according to the invention,

FIGS. 14a and 14b show a fifth device according to the invention.

### DETAILED DESCRIPTION OF THE EXAMPLES OF THE FIGURES

For the sake of clarity, the same reference numbers are used for some corresponding parts in different embodiments.

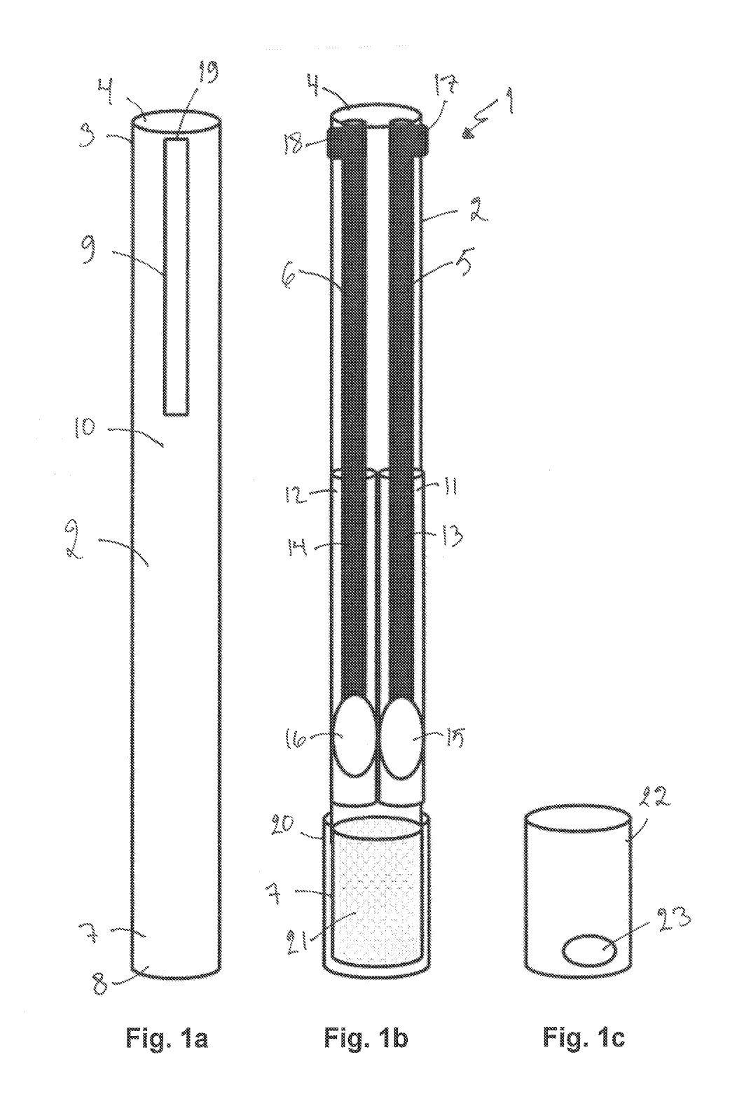

FIGS. 1a, 1b and 1c show some examples of possible parts of a device 1 for testing toxicity of a test sample. An elongated device body 2 protects and holds together different parts of the device 1. The device body 2 has a first end 3 with a swab opening 4 through which a control swab 5 and a test swab 6 are inserted into the device body. A second end 7 of the device body has vial fastening means 8 for releasably attaching a vial to the elongated body. Vial fastening means 8 can be e.g. threads or other fittings, such as a friction joint allowing a vial to be fastened to the device body. In normal use the first end 3 is the upper end of the device body 2 and the second end 7 is the lower end of the device body 2. One or more swab moving openings 9 are formed at the side 10 of the elongated device body. Swabs inside the device body 2 may be contacted and moved up and down inside the device body through the swab moving openings 9.

FIG. 1b shows one possible device 1 according to the invention. A control swab 5 and a test swab 6 are arranged inside swab tubes 11 and 12 inside the device body 2. The swab tubes 11 and 12 are arranged to keep the swabs in them separated from each other. The swab tubes 11 and 12 are open on their both ends and allow the swabs to move up and down inside the swab tubes. In this embodiment the lower ends of the swab tubes do not extend quite to the second end 7 of the device body. Swabs 5 and 6 comprise swab handles 13 an 14 and swab heads 15 and 16 arranged on one of the ends of the swab handles. Swab heads may be e.g. of polyester and swab handles may be e.g. of polypropylene. The swab head may be constructed to the swab handle without adhesives.

Levers or protrusions 17 and 18 are arranged on the other ends, i.e. upper ends of the swab handles 13 and 14. The levers 17 and 18 may be arranged to at least partly extend out through the swab moving openings 9 when the swabs are arranged inside the device body 2. This way the swabs 5 and 6 are easily contacted and moved up and down in the device 1 from outside of the device body 2, e.g. with a finger. If the upper ends 19 of the swab moving openings 9 are closed, like shown in FIG. 1a, the levers 17 and 18 hinder the swabs from accidentally dropping out of the device body. The swab handles 13 and 14 can be somewhat flexible, so that the allow bending of the handles when moving the swabs in and out through the swab opening 4.

FIG. 1b shows a vial 20 fastened at the vial fastening means 8, e.g. by mere friction between the vial 20 and the device body 2. A closed reconstitution liquid chamber 21 is positioned inside the vial 20, at the second end 7 of the device body, below the swab tubes 11 and 12. A grey shadowing represents an unpunctured, intact reconstitution liquid chamber 21.

FIG. 1c shows a vial 22 for a test reagent 23 such as freeze dried bacteria or other reagent capable of producing light. Such a vial may be used if the test reagent 23 is dry and needs to be reconstitution i.e. rehydrated prior to the tests.

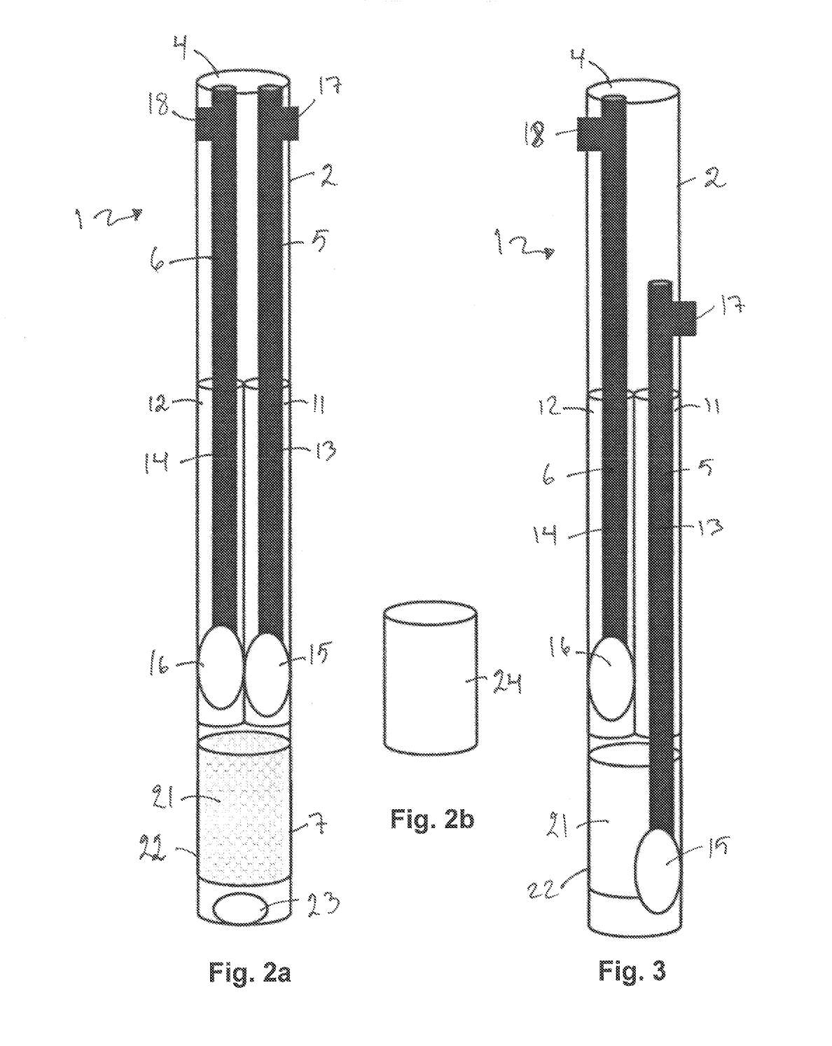

FIG. 2a shows one possible device 1 according to the invention. Here vial 22 with reagent 23 is attached on the device body 2, having replaced vial 20 of FIG. 1b. The reagent 23 is positioned below the reconstitution liquid chamber 21.

FIG. 2b shows a measurement vial 24. It may be attached at the second end 7 of the device body replacing other vials, before the device is inserted into a luminometer (not shown). The measurement vial is used to protect a luminometer i.e. the wet swabs will not touch the luminometer due to the measurement vial 24.

One possible use of the device 1 of FIG. 2a is described in FIGS. 3-8.

FIG. 3 shows how test reagent is reconstituted for the measurement by pressing the lever 17 and thereby the control swab 5 down to release the reconstitution liquid in the reconstitution liquid chamber 21. This allows the reconstitution liquid to mix with the reagent 23. The reconstitution liquid may also be introduced into the vial 22 in any suitable way, e.g. by pipeting. When the control swab 5 is pressed down it is moistened with the reconstituted test reagent. The control swab is lifted up and its swab head 15 contains the reagent for the test control.

According to the invention the reconstitution can also be done outside the device e.g. by pouring a known amount of the reconstitution liquid to a vial with the reagent 23 before attaching the vial to the device.

FIG. 4 shows how the test swab 6 is taken from the device body 2 through the swab opening 4 and the swab head 16 is moistened with the test sample in a test vial 25 outside the device body 2.

FIG. 5 shows how the test swab 6 is then returned to the test device body 2 through the swab opening 4. The lever 18 is pressed down until the swab head 16 reaches the reconstituted reagent in vial 22. The swab 6 is lifted up, the swab head 16 containing the mixture of the test sample and the reagent.

FIG. 6 shows the device 1 after the mixture of the test sample and reagent has been discarded and an empty vial, a measurement vial 24 is attached to the device body 2. The device contains now the control swab 15 with reagent only and the test swab 16 with reagent mixed with the test sample.

The device 1 can be incubated inside a luminometer or in an incubation chamber (not shown). The temperature for the control swab head 15 and test swab head 16 will be the same because they are inside the same device, close to each other and separated by a very thin layer of material only, the swab tubes 11 and 12. The device 1 comprises now liquid only in the swab heads 15 and 16, thus it may be turned or rotated and easily transported.

FIG. 7 shows how the control swab 5 is pressed down to its down position. Now the device 1 may be put in a luminometer. The swab head 15 is in the empty measurement vial 24 and luminescence of the control swab 5 is read. After the reading is done the device 1 is removed from the luminometer. The control swab 5 is lifted to up-position.

FIG. 8 shows how the test swab 6 is pressed down to its down position and the device may be put in a luminometer. Now the swab head 16 is in the empty measurement vial 24 and luminescence of the test swab is read. The device is removed from the luminometer.

If needed, the measurements can be repeated after different time intervals for both the control swab 5 and test swab 6. Now the device 1 can be discarded and the toxicity can be calculated.

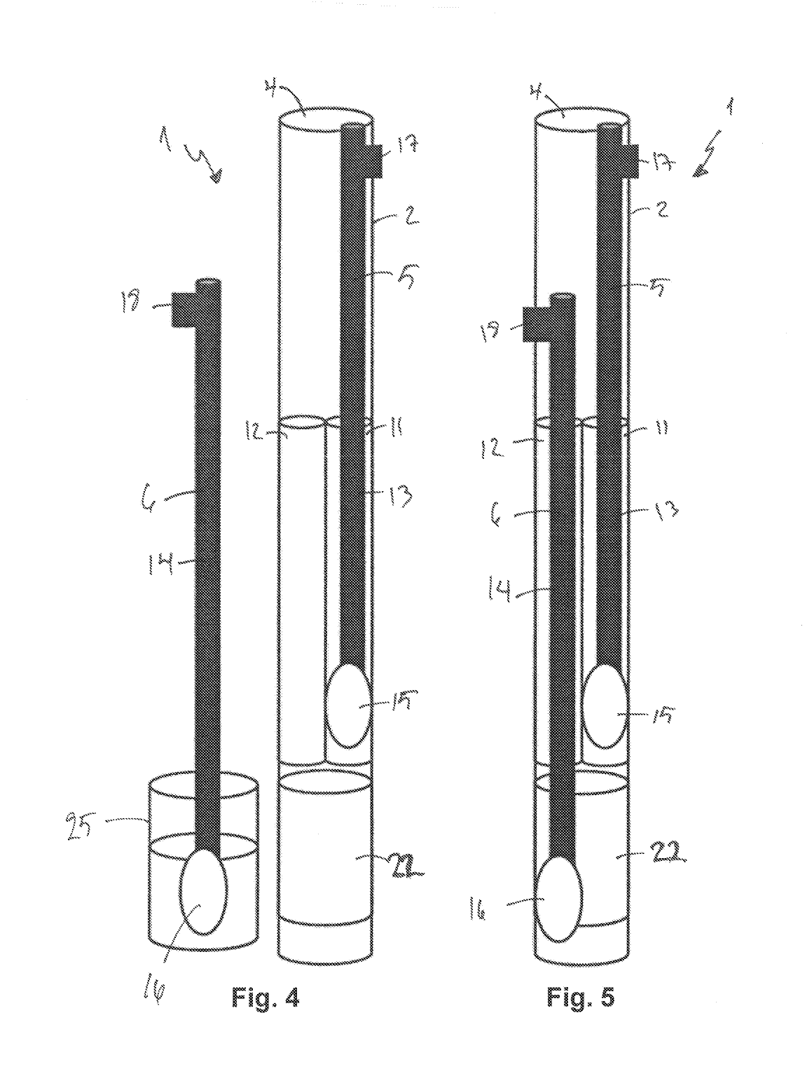

FIGS. 9a and 9b show a second device 101 according to the invention. The device 101 comprises an inner tube 102 and an outer tube 103. The inner tube is open at its upper end, i.e. it comprises a swab opening 104. Control and test swabs 105, 106 are in the inner tube. In this embodiment the inner tube 102 is approximately of the same length with or a little shorter than the swabs. In FIG. 9a the device 101 is not used and both swab heads 115 and 116 are pointing down. When a user takes a swab from the inner tube, she/he does not easily touch i.e. contaminate the swab head. FIG. 9b shows the situation after moistening the control swab 105 and the test swab 106 outside the device 101. The control swab is 105 inserted in the device with swab head 115 down, at the closed bottom 107 of the inner tube. The test swab 106 is inserted swab head 116 up, i.e. in such a way that the two swab heads point towards opposite directions. This way the moistened swab heads are not in contact with each other. No swab tubes inside the device are needed for separating the swabs due to the fact that the swab handles 113 and 114 are long enough. The outer tube 103 has been slid on to the inner tube 102. The outer tube 103 has a closed end 120 and an open end 121. The device 101 is thus closed and ready to be measured in a luminometer or transported e.g. to a laboratory.

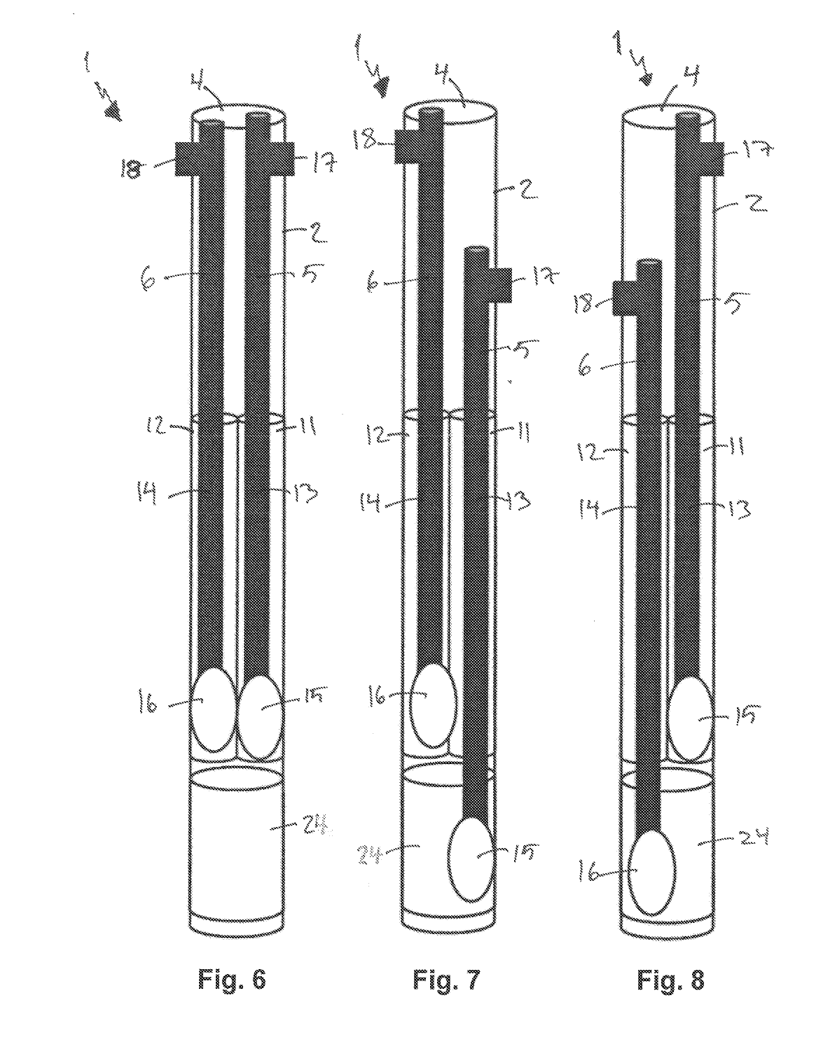

FIG. 10 shows a third device 101 according to the invention. It functions almost the same way as the device in FIGS. 9a and 9b, only now the device comprises only one swab 108 with swab heads 115 and 116 on both ends of the swab handle 109. One swab head functions as a control swab 115 and the other swab head as a test swab 116.

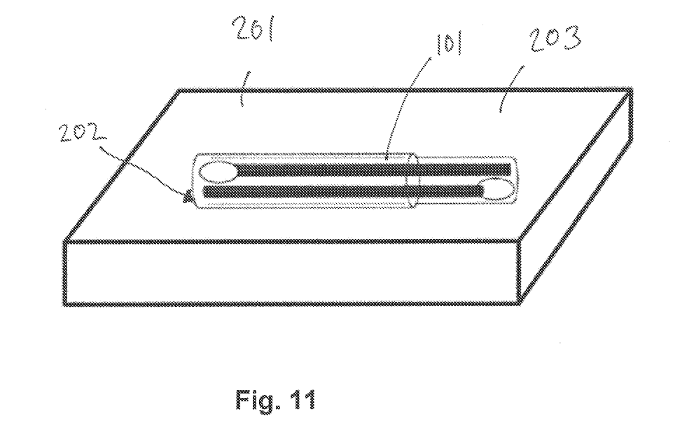

FIGS. 12a and 12b show a fourth possible device 301 according to the invention, FIG. 12b is a top view of the device in FIG. 12 a. A control swab 305 and a test swab 306 are arranged inside swab tubes 311 and 312 inside the device body, i.e. tube 302. The swab tubes 311 and 312 are arranged to keep the swabs in them separated from each other. The swab tubes 311 and 312 are formed by arranging a partition wall 350 in the middle of the device body tube 302. Swabs 305 and 306 comprise swab handles 313 an 314 and swab heads 315 and 316 arranged on one of the ends of the swab handles.

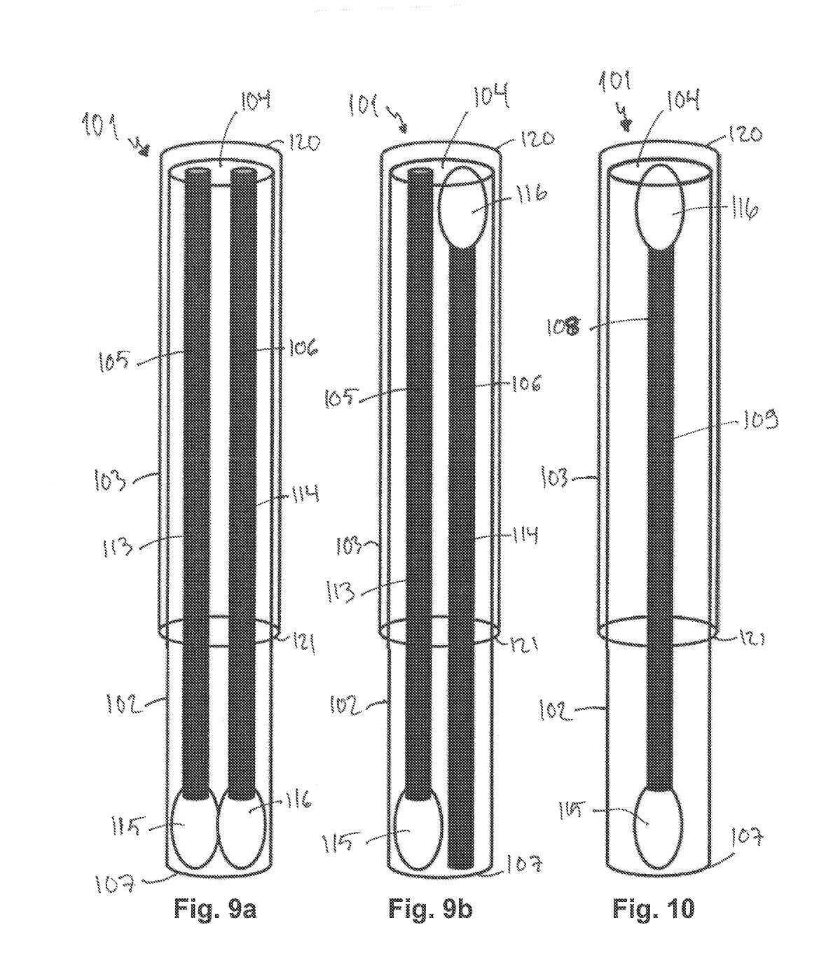

FIG. 11 shows a plate 201 or an insert for a plate reader luminometer. The outer diameters of the insert may be those of any typical plate in prior art, e.g. a 96-well microplate, i.e. a plate with 96 wells. Instead of the many wells in a normal plate, there is a larger opening or recess 202 on the top face 203 where a device 101 described in FIGS. 9a and 9b is inserted. An insert of the type presented in FIG. 11 may comprise one or more recesses adapted to receive any of the devices presented in FIG. 1 to 10 or 12.

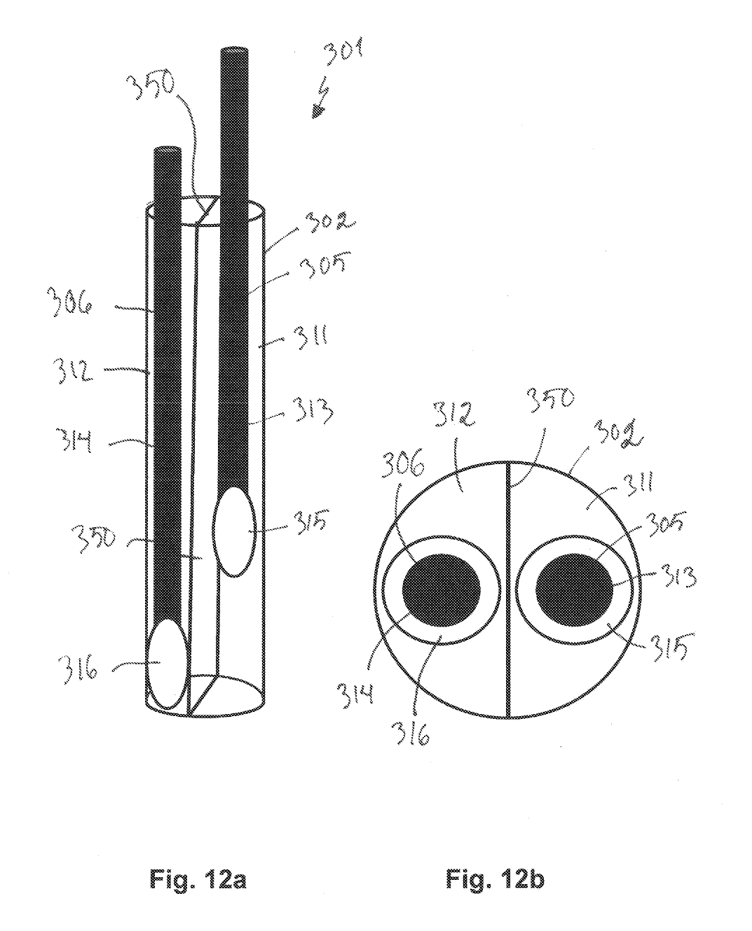

FIG. 13 shows an insert or a plate 401 of a plate reader according to the invention. The plate 401 comprises a top face 403 and a bottom face. A device according to the invention with swabs is arranged into one of the eight openings or recesses 402 of the plate 401 formed on the top face. Bottoms of the recesses 402 comprise light openings 405 or windows for passage of emitted light from a swab arranged in said recess to the bottom face side of the plate 401. It is possible to read luminescence from swab heads in a device according to the invention, such as a standard size cuvette, from the top face side and/or from the bottom through light opening 405. The adaptor plate may include aligning walls for aligning cuvettes. Non-transparent walls 410 are formed between recesses, e.g. at positions from B1 to B4.

In the embodiment of FIG. 13 the insert or a plate 401 has eight recesses in order to hold eight devices or cuvettes simultaneously. Each device or cuvette may contain two swab heads for the luminescence readings. In one embodiment of the invention, the reading positions are marked at the sides of top face 404. If the plate reader reads luminescence from the top face side, swab heads may be situated on any reading position, e.g. A1-A4, C1-04, E1-E4, G1-G4, B9-B12, D9-D12, F9-F12, H9-H12.

The light openings 405 at the bottom face 404 are situated at A1, C1, El, G1, B12, D12, F12 and H12. If a device or cuvette in a recess has a swab head positioned at these reading positions, the plate reader can read its luminescence value from the bottom face side. Thus, if capable, a plate reader may read luminescence from both sides of the plate 401.

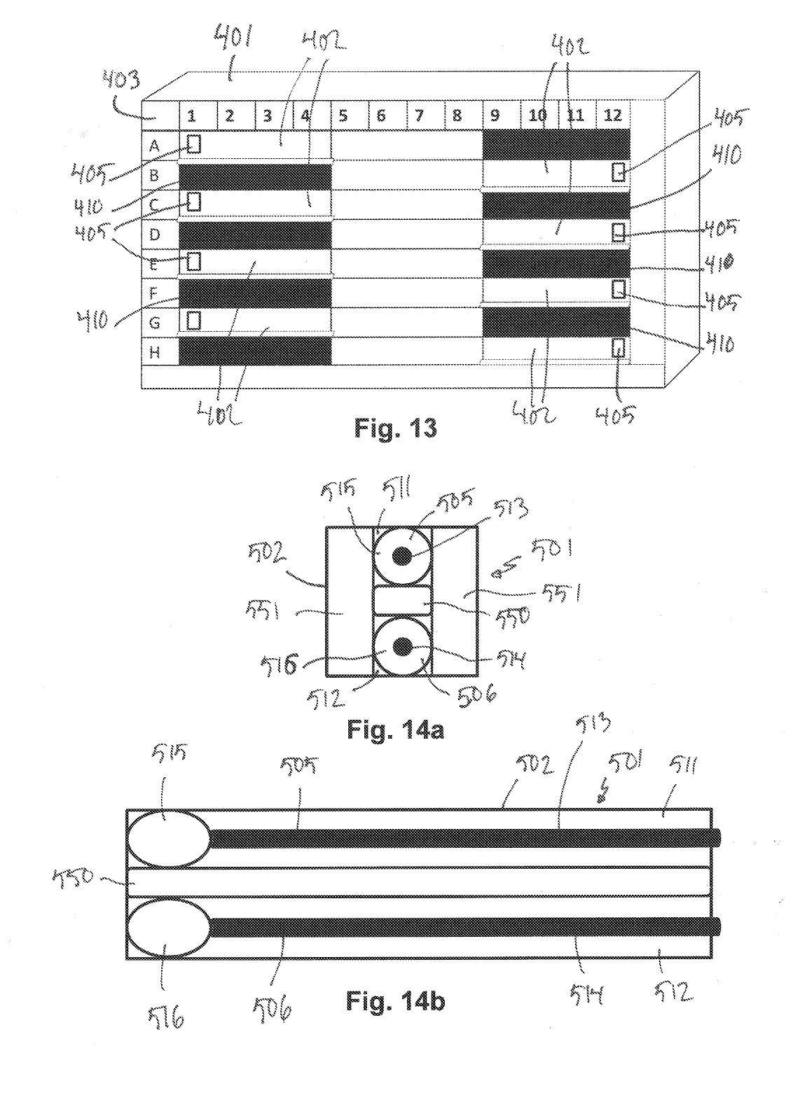

FIGS. 14a and 14b show a fifth possible device, a rectangular cuvette 501 according to the invention. FIG. 14a is a cross-sectional view of the device in FIG. 14b. Two swabs, e.g. a control swab 505 and a test swab 506 are arranged inside swab tubes 511 and 512 inside the device body, i.e. the outer walls 502 of cuvette 501. The swab tubes 511 and 512 are arranged to keep the swabs in them separated from each other. The swab tubes 511 and 512 are separated by a non-transparent partition wall 550 in the middle of the device body tube 302. Swabs 505 and 506 comprise swab handles 513 and 514 and swab heads 515 and 516 arranged on one of the ends of the swab handles. The cuvette 501 is formed such that it fits into a recess of an insert or a plate 201 or 401 presented above. The dimensions of the cuvette 501 and swabs may be arranged such that the swabs do not move inside the cuvette during the measurement cycle inside the luminometer or during the incubation. The cuvette may also comprise other parts, e.g. the cuvette 501 comprises empty spaces or walls 551.

In one embodiment of the invention, the control swab 505 is on one side of the non-transparent partition wall 550 and the test swab 506 is on the other side. Due to the non-transparent partition wall 550, luminescence from the swabs can be measured from both sides simultaneously. If the plate reader is capable of reading luminescence from one side only, the cuvette 501 or the plate 401 needs to be turned upside down for reading luminescence of the swab on the other side of partition wall 550.

The examples shown present the light producing test reagent as dry, needing reconstitution before the swab heads are moistened with it. Naturally it is possible to use a test reagent in liquid form from the beginning. It that case no reconstitution liquid is needed in the device.

The figures show only a few preferred embodiments according to the invention. Facts of secondary importance with regards to the main idea of the invention, facts known as such or evident for a person skilled in the art, such as power sources or support structures possibly required by the invention, are not necessarily separately shown in the figures. It is apparent to a person skilled in the art that the invention is not limited exclusively to the examples described above, but that the invention can vary within the scope of the claims presented below. The dependent claims present some possible embodiments of the invention, and they are not to be considered to restrict the scope of protection of the invention as such.

1. A method for testing toxicity of a test sample, the method comprising at least following steps:
providing an aliquot of light producing test reagent liquid in a reagent vial;
moistening a control swab with the light producing test reagent liquid;
moistening a test swab with the test sample;
moistening the test swab with the light producing test reagent liquid;
reading luminescence of the control swab in a luminometer and thereby obtaining a luminescence value of a control sample;
reading luminescence of the test swab in the luminometer and thereby obtaining a luminescence value of the test sample;
calculating the toxicity of the test sample by comparing the luminescence value of the control sample to the luminescence value of the test sample;
wherein the moistenings of the control swab and the test swab with the light producing test reagent liquid are done conducted with the same aliquot of light producing test reagent liquid in the same reagent vial.

2. The method according to claim 1, wherein
the test sample and the aliquot of light producing test reagent liquid are mixed together, thereby producing a mixture, and thereafter
the test swab is moistened with the test sample and with the light producing test reagent liquid by moistening the test swab in said mixture.

3. The method according to claim 1, wherein
the aliquot of light producing test reagent liquid in the reagent vial is provided by reconstituting a light producing test reagent with reconstitution liquid in the reagent vial.

4. The method according to claim 3, wherein the light producing test reagent is reconstituted by puncturing a reconstitution liquid chamber with the control swab, thereby arranging the reconstitution liquid to mix with the light producing test reagent in the reagent vial.

5. The method according to claim 1, wherein the test is conducted in an elongated device body having
a first end with a swab opening for inserting the control swab and/or the test swab into the device body and for removing them from the device body, and
a second end with vial fastening means for releasably attaching a vial to the elongated body;
whereby the method further comprises attaching to and/or removing from the vial fastening means at least one of the following:
the reconstitution liquid chamber;
the reagent vial containing test reagent liquid;
an empty reagent vial;
a vial containing dried light producing test reagent
a measurement vial for protecting the luminometer during the reading of luminescence; and inserting at least part of the control swab and/or the test swab in to a vial attached to the vial fastening means.

6. The method according claim 5, wherein the control swab and the test swab are moved inside the elongated device body in their own separate tubes keeping the swabs separated from each other.

7. The method according claim 6, wherein moving the control swab and the test swab inside the elongated device body is conducted by contacting them through one or more swab moving openings formed at the side of the elongated device body.

8. The method according claim 1, wherein the light producing test reagent is a natural or genetically modified luminescent organism, selected from: freeze dried organism, liquid dried organism or fresh organism.

9. The method according claim 1, is made directly from the swabs.

10. The method according claim 9, wherein substantially all liquid present is absorbed in the swabs during the reading of luminescence of the control swab and the test swab.

11. The method according claim 1, wherein both the control swab and the test swab comprise a swab head made of 100% polyester.

12.-16. (canceled)

17. The method of claim 8, wherein the organism is light producing bacteria or yeast.

18-19. (canceled)

20. The method of claim 17, wherein the bacteria is Aliivibrio fischeri.
